(19) United States
(12) Patent Application Publication
(10) Pub. No.: US 2008/0177142 A1
(43) Pub. Date: Jul. 24, 2008

(54) ENDOSCOPE WITH DILATING INSERTION TUBE

(75) Inventor: Willliam Roskopf, Pleasanton, CA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: BOSTON SCIENTIFIC SCIMED, INC., Maple Grove, MN (US)

(21) Appl. No.: 12/016,768

(22) Filed: Jan. 18, 2008

Related U.S. Application Data
(60) Provisional application No. 60/886,075, filed on Jan. 22, 2007.

(51) Int. Cl. A61B 1/01

(52) U.S. Cl. 600/115

(57) ABSTRACT

An endoscope has an insertion tube with a nontapered or tapered dilator. The dilator may be inflated via a control handle connected to the insertion tube. The endoscope and insertion tube may be used to examine a patient and to relieve a blockage with the use of the insertion tube.

### CROSS REFERENCE TO RELATED APPLICATION

This application claims the benefit of U.S. Provisional Application No. 60/886,075, filed on Jan. 22, 2007.

### BACKGROUND

As an aid to the early detection of disease, it has become well established that there are major public health benefits from regular endoscopic examinations of internal structures, such as the alimentary canals and airways, e.g., the esophagus, lungs, colon, uterus, bronchi and other organ systems. A conventional imaging endoscope used for such procedures comprises a flexible tube with a fiber optic light guide that directs illuminating light from an external light source to the distal tip where the light exits the endoscope and illuminates the tissue to be examined. Frequently, additional optical components are incorporated to adjust the spread of the light exiting the fiber bundle and the distal tip. An objective lens and fiber optic imaging light guide communicating with a camera at the proximal end of the scope, or an imaging camera chip at the distal tip, produce images that are displayed for viewing by the physician. In addition, most endoscopes include one or more working channels and lumens through which fluids, or medical devices, such as biopsy forceps, snares, fulguration (electrocauterization) probes, and other tools, may be passed.

The esophagus and other body lumens may periodically become restricted or blocked due to any number of conditions. For example, a blockage or stricture of the esophagus may limit the passage of food and fluids and may endanger a patient's well being. To alleviate a blockage, a physician may perform a visual examination of the blockage using an endoscope. The physician can then determine how to treat the condition, for example, by resorting to surgery or dilation. Once the cause of the blockage is determined, dilation may be indicated. Dilation is conventionally performed by a variety of means including, but not limited to, balloon dilation, savary dilation, Maloney dilation, and metal olives. A commonly used dilator is a flexible, elongated device of increasing diameter, generally resembling an elongated thin cone. Dilators may come in tapered or nontapered versions and in different sizes. The dilator is slowly advanced through the blockage until dilation is achieved. One problem with this type of dilator is that the dilator has to be removed, and an endoscope will need to be intubated to enable the physician to view the results of the dilation. If the results are not acceptable, the procedure is repeated until satisfactory results can be verified by viewing with the endoscope.

### SUMMARY

In view of the multiple steps necessary with the prior art dilators and endoscopes, embodiments of the present invention are related to an endoscope with an integrated dilator that may be used to dilate a blockage or stricture of a body lumen and then verify the results without removal of the dilator from the patient because the endoscope combines a dilator with the capacity to view images inside of the body lumen.

In one embodiment, the endoscope includes an insertion tube having an expandable outer diameter, such as an expandable sheath that covers the insertion tube. The sheath defines an expandable chamber that may be selectively enlarged to produce a tapered or nontapered profile or any other profile along the insertion tube that functions as the dilator. The operator of the endoscope or physician may control the outer diameter of the insertion tube by introducing or withdrawing an inflation fluid, air, or gas to and from the chamber. In another embodiment, the insertion tube has an outer sheath that can be inflated with a gas or liquid to create a dilator. In another embodiment, the sheath may be expanded via mechanical means, such as with an internal cage (or stent) without inflation. The sheath may have elastic or shape memory elements that expand to perform dilation.

A method of dilating is described using an endoscope having an insertion tube with an expandable outer diameter. The insertion tube is delivered to a region of interest and the outer diameter of the insertion tube is enlarged to convert the insertion tube into a dilator while retaining visual capability in order to view the results of dilation without having to withdraw the dilator and reintubate the body with an endoscope every time the results are desired to be viewed.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is an illustration of an endoscopic system with an insertion tube having a tapered outer diameter in accordance with an embodiment of the present invention;

FIG. 2 is a cross-sectional illustration of an insertion tube having an expandable outer diameter in accordance with an embodiment of the present invention;

FIG. 3 is a schematic block diagram of an endoscopic system with an insertion tube having an expandable outer diameter in accordance with an embodiment of the present invention;

FIG. 4 is an illustration of a control handle to control an endoscopic system with an insertion tube having an expandable outer diameter in accordance with an embodiment of the present invention;

FIG. 5 is an illustration of a portion of the insertion tube having an expandable outer diameter in accordance with an embodiment of the present invention;

FIG. 6 is an illustration of a portion of the insertion tube having a tapered outer diameter in accordance with an embodiment of the present invention;

FIG. 7 is an illustration of a portion of the insertion tube having an expandable outer diameter in accordance with an embodiment of the present invention;

FIG. 8 is an illustration of a portion of the insertion tube having a nontapered outer diameter in accordance with an embodiment of the present invention;

FIG. 9 is an illustration of a cylinder having an expandable outer diameter in accordance with an embodiment of the present invention;

FIG. 10 is a cross-sectional illustration of a portion of the insertion tube having an expandable outer diameter in accordance with an embodiment of the present invention;

FIG. 11 is an illustration of a portion of the insertion tube having a tapered outer diameter in accordance with an embodiment of the present invention;

FIG. 12 is an illustration of a portion of the insertion tube having a rounded profile in accordance with an embodiment of the present invention;

FIG. 13 is an illustration of a portion of the insertion tube having an increasing tapered outer diameter in accordance with an embodiment of the present invention;

FIG. 14 is an illustration of a portion of the insertion tube having a combination of a decreasing taper, a constant diameter, and an increasing taper in accordance with an embodiment of the present invention; and

FIG. 15 is an illustration of a portion of the insertion tube having a tapered outer diameter in accordance with an embodiment of the present invention.

### DETAILED DESCRIPTION

As indicated above, embodiments of the invention relate to medical systems for use in imaging an internal body lumen or passageway and dilating a constriction therein. Embodiments of the invention include endoscopes, imaging catheters and other visualization tools including an integrated dilator. In one embodiment, an endoscope may include an insertion tube having a tapered or nontapered outer diameter, or, alternatively, an expandable outer diameter. An expandable outer diameter may increase radially from a first diameter to a second relatively larger diameter along a length of the endoscope's insertion tube. The increased outer diameter may form a taper that increases from a distal location to a proximal location on the insertion tube or vice versa. Alternately, the diameter may be nontapered. A nontapered outer diameter is one that is substantially constant from a distal location to a proximal location. Other embodiments may include a combination of increasing tapers, decreasing tapers, nontapers, or rounded profiles, created by expansion of an outer sheath, either through inflation or through the use of a mechanical cage, stent, or shape memory material.

In one embodiment, the outer diameter of the insertion tube may be increased to provide the dilating action via a controller under the direction of a physician. In another embodiment, the outer diameter of the insertion tube may also be decreased from an increased outer diameter to a relatively smaller diameter for insertion and removal as selected by the physician.

An endoscopic system having an insertion tube with a dilating action may be used for a variety of different diagnostic and interventional procedures, including colonoscopy, upper endoscopy, bronchoscopy, thoracoscopy, laparoscopy, ureteroscopy, hysteroscopy, or other procedure where constrictions may be encountered. Although exemplary embodiments are described hereinafter with reference to endoscopes, it will be appreciated that aspects of the invention have wide application, and may be suitable for use with other medical devices, such as catheters or other imaging devices. Accordingly, the following description and illustrations should be considered illustrative in nature and not limiting.

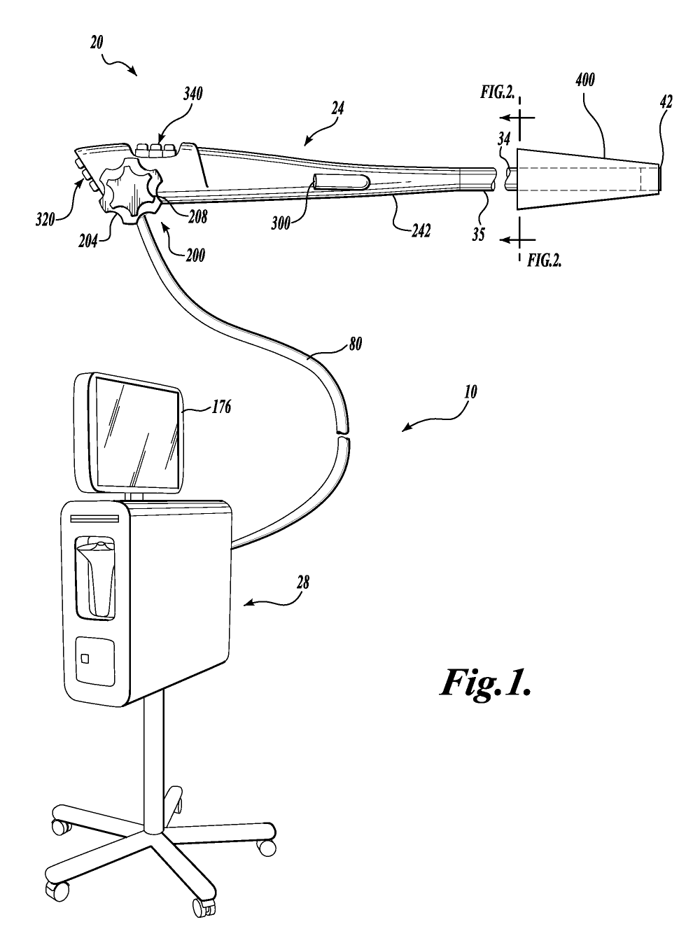

FIG. 1 illustrates one exemplary embodiment of an endoscopic video imaging system 10 that may be used to dilate a blockage in a body lumen. The system 10 includes an imaging endoscope 20 having a control handle 24, an insertion tube 34, and a communications conduit 80. The system 10 further includes a control cabinet 28 connected to the endoscope 20 via a proximal connector (not shown) at the end of the communications conduit 80. The insertion tube 34 extends from a proximal location 35 or end to a distal location 42 or end. Along any length of the insertion tube 34, between or from the proximal end 35 and the distal end 42, the insertion tube's outer diameter may be increased and decreased to form a tapered or nontapered dilator. In FIG. 1, for example, the insertion tube 34 is illustrated having a tapered dilator 400. However, other embodiments of insertion tube 34 may have a nontapered dilator 400. The dilator may retain its shape in the expanded condition or as explained in further detail below may be selectively expandable. For example, in one embodiment, the dilator may be made of a rubber or foam like material that allows it to be compressed by an external instrument such as a sleeve (not shown) that is slidable over the shaft. Upon removal of the sleeve, the dilator may expand to its maximum outer diameter. In some embodiments, it may be desirable to place a sloping edge on the proximal end of the dilator so that the sleeve can be fitted over the dilator. In another embodiment, the dilator could be held constricted with bands, for example. Still, in another embodiment, sections of the dilator may be expandable to perform as a balloon-type dilator.

The endoscope 20 may be functionally controlled by the physician from the control handle 24. The control cabinet 28 provides image processing capabilities, as well as supplies power, fluids, air, water, suction, etc., to various devices and lumens within the endoscope 20. The endoscope 20 and insertion tube 34 may be flexible, partially-flexible, or rigid. The endoscope 20 may be of the optical type (i.e., a fiberseope) in which an optical image is carried on a coherent fiber optic bundle, or the video type, in which a miniature imaging sensor, such as a charge coupled device (CCD) or CMOS imaging sensor, is disposed at or adjacent the distal end 42 of the insertion tube 34.

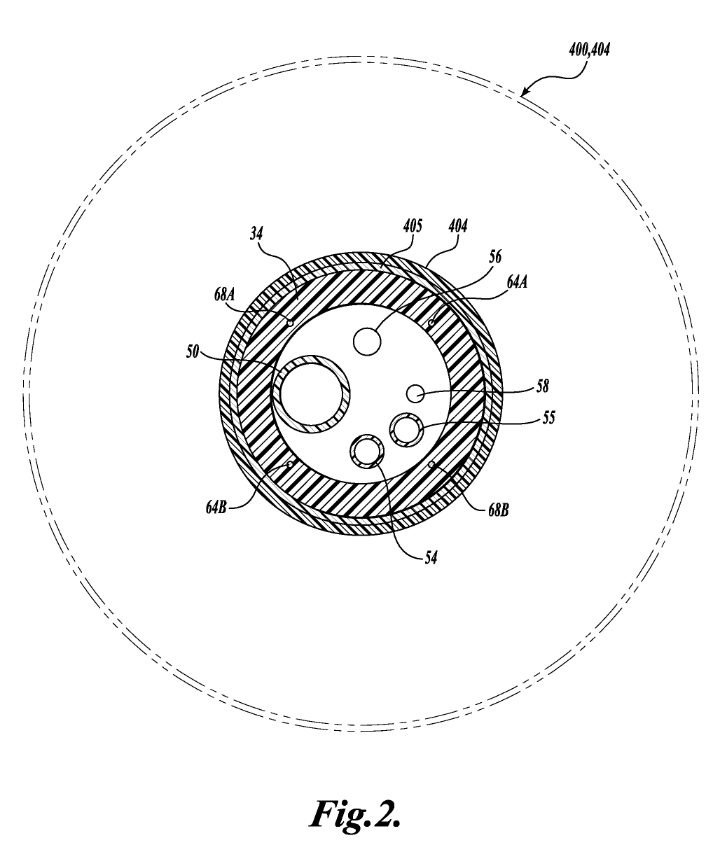

The insertion tube 34 may further include one or more lumens concealed within the interior of the insertion tube 34 for the purpose of performing endoscopic procedures and for the purpose of delivering or extracting fluids, liquids and gases, and/or medical devices into and out of the body. For example, as illustrated in FIG. 2, the insertion tube 34 may include one or more working channels 50, a lens wash and/or insufflation lumen 54, a jet wash lumen 55, and, optionally, a suction lumen (not shown). The insertion tube 34 may also include one or more electrical cables 56 and 58 for use in supplying power to illumination LEDs, timing and control signals to an imaging sensor, and for transmitting image signals back to the control cabinet 28, etc. Alternatively, fiber optic cables may be provided for sending the image signals. One or more of the lumens and/or electrical cables may extend from the distal end 42 of the insertion tube 34 through the control handle 24 to the proximal connector at the proximal end of the communications conduit 80. These lumens may extend along the whole length of the tube or partially along the length of the tube. Additionally, the lumens may have exit and entry ports at the ends or on the sides of the insertion tube 34. Finally, the endoscope 20 may include at least one pair of control wires 64A and 64B, and preferably two pairs of control wires 64A and 64B, and 68A and 68B that are secured at or adjacent to the distal end 42 of the insertion tube 34 and that terminate at their proximal ends in the control handle 24. Upon rotation of the knobs 204, 208 on the control handle, the pairs of wires 64A, 64B, 68A, 68B are selectively tensioned to steer the distal tip in a desired direction.

In the embodiment as illustrated in FIG. 2, the insertion tube 34 includes a selectively expandable outer sheath 404 that either inflates or expands to form the dilator 400. The insertion tube 34 has an inner sheath 405 that covers the insertion tube's 34 body and is located underneath the outer sheath 404 and is used to create a pressure-tight chamber. Compared to the inner sheath 405, the outer sheath 404 may only extend for a portion of the length of the insertion tube 34. Outer sheath 404 may be elastically expanded to define the dilator 400. It is to be appreciated that the diameter of the dilator 400 may not be constant along the length of the insertion tube 34, but may taper from the distal end 42 toward the proximal end 35 or vice versa. Alternatively, the dilator 400 may have a uniform diameter along its length. The remainder of the insertion tube 34 that does not form the dilator 400 maintains a smaller diameter relative to the diameter of the dilator 400 in its expanded state. In other embodiments, the dilator may have variable profiles, including combinations of tapering toward the proximal end, tapering toward the distal end, constant diameter, rounded profiles (balloon-shaped), and combinations of all the above, or alternatively different profiles.

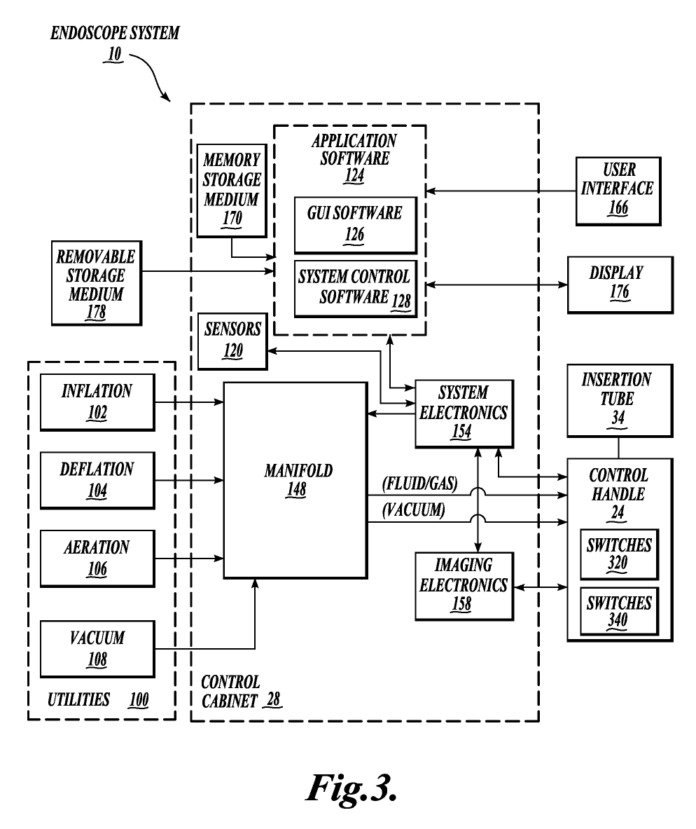

FIG. 3 is a block diagram of the endoscopic system 10, including one exemplary embodiment of the control cabinet 28. The control cabinet 28 is preferably mounted on wheels allowing mobility to enable placement of the control cabinet 28 near a patient prior to an examination procedure. The control cabinet 28 supplies power to the endoscope 20 from a source of electrical power, either alternating current (AC) or direct current (DC), as well as controls the delivery of one or more utilities 100, including, for example, an inflation gas or liquid 102, an insufflation air or gas 104, and vacuum 108. The inflation fluid, which may be gas, liquid, or semi-liquid, is selectively delivered to a lumen of the endoscope to inflate the dilator 400 on the insertion tube 34. The deflation source 104 provides a pressure below the pressure within dilator 400 to withdraw the inflation fluid from the dilator 400. Alternatively, deflation of the dilator 400 may be carried out by releasing the inflation fluid through a vent in communication with the interior of the dilator 400 to vent the fluid out of dilator 400 either directly into the body lumen, to the atmosphere, or to a lumen within the endoscope. Additionally, or alternatively, the dilator may be vented by allowing the fluid in the dilator to drain under the force of gravity. Such venting techniques have the advantage of simplicity in design and obviates the need for a separate deflation source to deflate the dilator 400. The aeration source 106 is pressurized gas, such as compressed air. The vacuum source 108 provides a pressure below atmospheric pressure. Vacuum source 108 may provide another method for deflation of dilator 400. Deflation by vacuum source 108 may take place through a vacuum collection jar or a tube directly connected to the vacuum source 108. Fluids, air and vacuum are produced by pumps that may be internal or external to the control cabinet 28. The utilities 100 may enter a manifold 148 containing appropriate valves and controls to route utilities 100 through control cabinet 28 and ultimately to lumens within insertion tube 34 as selected. The manifold 148 is functionally controlled by the system electronics 154, also contained within the control cabinet 28. Manual controls in the form of switches 340 on the control handle 24 are connected to the system electronics 154, and are functional to operate the delivery of utilities 100 to the insertion tube 34. The manifold 148 may be located externally or internally with respect to the control cabinet 28. In one embodiment, the manifold is located external to the control cabinet 28; however, actuators that open or close valves on the manifold may be located internally within the control cabinet 28.

The control cabinet 28 may include additional components and functionality to control the operation of the endoscope including a suite of application software 124 including a graphical user interface (GUI) software application 126 and a system control software application 128. In addition, the control cabinet 28 may include an imaging electronics board 158. GUI software application 126, acting with the imaging electronics board 158, provides the physician with live endoscopic video images on a display 176 using control cabinet user interface 166 or GUI navigational controls 320 on the control handle 24. System control software application 128 is the central control program of application software 124 that receives input from the user interface 166 or control handle 24, and provides system software control for the features and functions necessary to operate the endoscopic imaging system 10 and to deliver the appropriate utilities 100 to the insertion tube 34.

It will be appreciated that the configuration of the control cabinet 28 described above and shown in FIG. 3 is exemplary. Accordingly, the control cabinet 28 may have many other configurations and/or features.

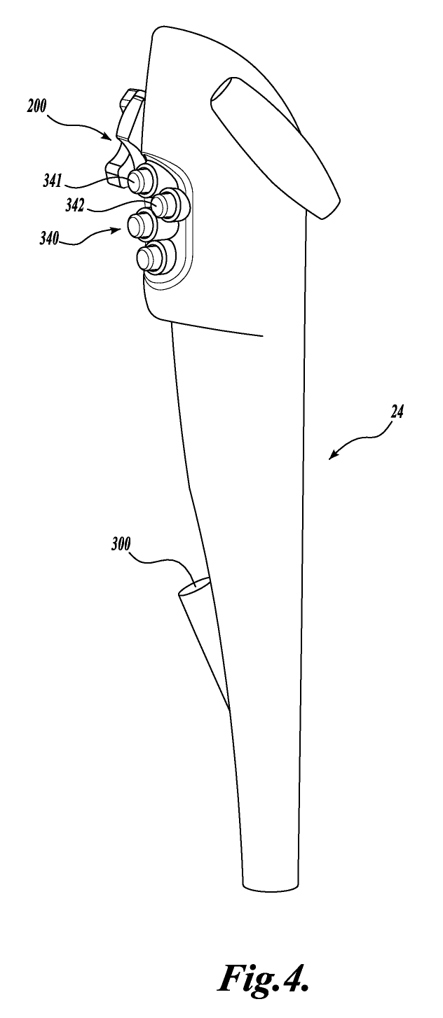

FIG. 4 illustrates one exemplary embodiment of a control handle 24. The control handle 24 includes an entrance port 300 formed in the exterior surface of the control handle housing. The entrance port 300 provides access to the working channel 50 described above in relation to FIG. 2. The control handle 24 includes sets of switches 320 (see FIG. 2) and 340 for GUI navigational control, delivery of utilities to insertion tube 34, and controlling the dilator. Inflation 102 and deflation 104 utilities are for inflating and optionally for deflating the dilator 400 disposed along the length of the insertion tube 34 on command.

As shown in FIG. 4, the control switches 340 may be positioned in an ergonomic arrangement on the control handle 24 and may be actuated by manual depression. The control switches 340 are in electrical communication with the appropriate system component in the control cabinet 28, such as the application software 124, through electrical cables that are routed through the communications conduit 80. Alternatively, the control signals from the switches may be transmitted wirelessly or optically. Control switches 340 may be located along the side of the control handle 24 and include, for example, an inflation switch 341 and a deflation switch 342. Other switches may include a vacuum bolus wash switch, a jet wash switch, and a lens wash switch. The inflation switch 341 may activate the delivery of an inflation fluid at the lumen in the insertion tube 34 to cause the insertion tube's outer sheath 404 to expand and increase in diameter. The inflation fluid may fill a chamber that is defined between the outer sheath 404 and the second, inner sheath 405 that surrounds the insertion tube's 34 body. The deflation switch 342 activates the withdrawal of the inflation fluid from the chamber to decrease the outer diameter of the insertion tube 34 and return the inflation fluid to a reservoir. Switches include “soft” switches (i.e., sensitive to touch or heat) and “hard” switches, such as buttons, toggle switches, rotary knobs, etc. In still other embodiments, activation of the inflation or expansion of the dilator may be accomplished manually and without the need for electronic controls and software. For example, the inflation fluid can be pumped into the dilator using a syringe or a hand pump.

FIGS. 5 and 6 illustrate one embodiment of an insertion tube 34 with a dilator 400. The endoscope 20 having a dilatable insertion tube 34 allows dilation of body lumens that have become blocked while maintaining a view of the surrounding tissue. In one embodiment, a dilatable insertion tube 34 for an endoscope 20 may be provided by attaching an elastic, expandable outer sheath 404 to an underlying sheath 405 that surrounds the insertion tube's 34 body. The purpose of outer sheath 404 is to expand into the dilator 400 along a section of the insertion tube 34. The sheath 404 has a distal end 416 and a proximal end 418. The distal end 416 of the outer sheath 404 may be circumferentially welded to, or attached in a pressure-tight manner, to the sheath 405 at the distal end 42 of the insertion tube 34. The proximal end 418 of the sheath 404 may be circumferentially welded to, or otherwise attached in a pressure-tight manner, to the sheath 405 at a location that is proximal from the distal end 42 of the insertion tube 34, but may not extend completely to the proximal end 35 of the insertion tube 34. The sheath 404 and sheath 405, therefore, may define a pressure-tight chamber 414 therebetween that has approximately no volume when deflated. In this condition, the insertion tube 34 and deflated dilator 400 may be easily intubated into a patient. The chamber 414 may be flexible to prevent restricting the movements of the distal end 42 of the insertion tube 34. A lumen 55 within the insertion tube 34 is connected to the inflation source 102 and/or to the deflation source 104, described above. Lumen 55 serves to deliver the inflation fluid or gas to the chamber 414 through an opening 412 in the sheath 405 that leads into the chamber 414. In some embodiments, the lumen 55 may be routed on the outside of the insertion tube 34. The inflation fluid or gas fills the chamber 414 and causes expansion of the sheath 404 and an increase in the insertion tube's 34 outer diameter. The remainder of the insertion tube 34 not covered by sheath 404 retains the relative smaller diameter. The sheath 404 may be fabricated to expand in a predetermined manner to produce a tapered dilator 400 or a nontapered dilator or any other desired profile.

FIG. 6 illustrates the inflated tapered dilator 400 created by filling the chamber 414 with inflation fluid. Inflation/deflation of chamber 414 is achieved through actuation of the inflation/deflation switches 341, 342, respectively, on control handle 24, described above. When inflated, the dilator 400 is suitable to perform dilation of a body lumen. The material used to construct the sheath 404 has sufficient elasticity to expand into the shape as desired, and also to contract to lie adjacent to the sheath 405 when the chamber 414 is evacuated. Materials for the sheath 404 may include, but are not limited to, elastomeric polymers such as a polyurethane, silicone, latex, or a high strength thermoplastic elastomer, such as a polyether block amide (such as Pebax®).

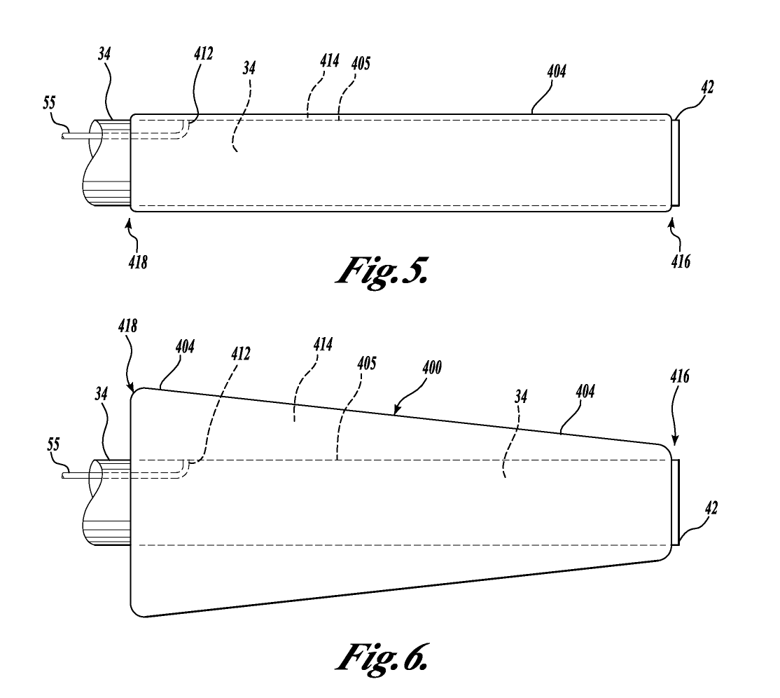

FIGS. 7 and 8, by way of comparison to FIGS. 5 and 6, illustrate an embodiment wherein the outer sheath 404 has been omitted from the insertion tube 34 to provide a flush surface along the insertion tube's 34 body to facilitate intubation and removal. Omitting the sheath 404 eliminates a possible bump that may be created by the weldment of the outer sheath 404 to the inner sheath 405. In this embodiment, at least a portion of the inner sheath 405 surrounding the insertion tube's 34 body is expandable to define the dilator 400. An area surrounding the expandable portion of the sheath 405 is sealed in a pressure-tight manner so that pressurization of the insertion tube in that area causes the sheath 405 to expand radially outward. For example, in many endoscopes, the outer sheath includes an outer jacket having a braided mesh therein to provide increased torque transfer. The braid can be left out of the sheath in an area and supported underneath with a cylindrical support 430. Inflation of the area between the jacket and the support will cause the outer diameter of the endoscope to increase and form a dilator. In some embodiments, the entire insertion tube may be sealed so that it can be pressurized. In alternative embodiments, a smaller portion of the insertion tube may be sealed.

In another embodiment, the sheath 405 is circumferentially attached in a pressure-tight manner at a proximal location 418 on the insertion tube 34. For example, the sheath 405 can be formed of a flexible material that is attached to the insertion tube 34 with an adhesive from the proximal end 35 of the insertion tube 34 to the proximal location 418. Adhesive is omitted between the proximal location 418 and the distal end 416 of sheath 405. Adhesive may be applied circumferentially at the distal end 416 to seal the sheath 405 circumferentially to the insertion tube's 34 distal end 42. The sheath 405, therefore, can form a pressure-tight chamber 414a where the adhesive was omitted that is defined by a section of the sheath 405 and an underlying cylindrical support 430 that prevents inflation fluid from migrating into the interior of the insertion tube 34. Inflation fluid may be supplied through the lumen 55 to discharge at the opening 420 under the sheath 405 and between the distal end 416 and the proximal location 418. Fluid may be introduced underneath the sheath 405 so that the sheath 405 may expand to create a nontapered dilator 400a, as illustrated in FIG. 8. The sheath 405, being circumferentially attached at the distal end 42 and the proximal location 418, may prevent the sheath 405 from expanding beyond these boundaries. The remaining section of the insertion tube 34 outside of these boundaries is not inflatable and retains its initial diameter.

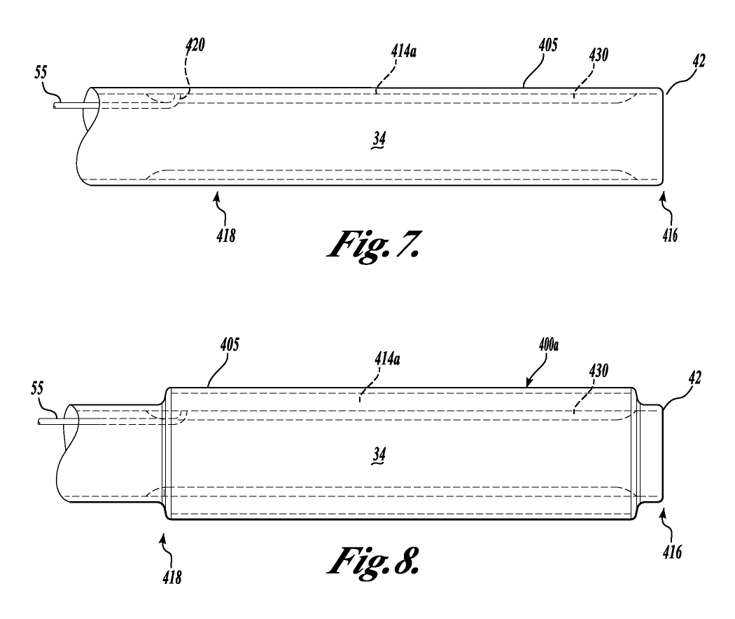

Referring to FIG. 9, another embodiment of achieving a dilatable insertion tube 34 for the endoscope 20 is illustrated. In this embodiment, a dilatable insertion tube 34 for an endoscope 20 may be provided by placing an inflatable sleeve 500 over the insertion tube 34. The sleeve 500 is defined by an inner wall 506 and an outer wall 504 to form a cylinder. The inner wall 506 may lie adjacent to the outer sheath of the insertion tube 34. Outer wall 504 may be made from an elastic material, described above, to expand and to cause an increase in the sleeve's 500 outer diameter. The inner wall 506 defines a central cavity 510 in which insertion tube 34 may fit. Inner wall 506 and outer wall 504 define a chamber 514 therebetween. Chamber 514 may receive an inflation fluid or gas. Walls 504 and 506 may be circumferentially joined in a pressure-tight manner at the distal end 516 and at the proximal end 518 of the sleeve 500. The sleeve 500 may include a base 502 at the proximal end 518. The sleeve 500 may be connected to the insertion tube 34 of endoscope 20 via the base 502. To this end, the base 502 may include a threaded connector (not shown), or an interlocking structure (not shown), to affix the sleeve 500 to the insertion tube 34. Alternatively, an adhesive may be used on the inner wall 506 to adhere the inner wall 506 to the insertion tube 34. The base 502 includes an aperture 508 through which the insertion tube 34 of endoscope 20 may pass through. Sleeve 500 does not necessarily extend the entire length of insertion tube 34. Sleeve 500 may be attached at any location along the length of the insertion tube 34. Additionally, sleeve 500 includes an inlet/outlet port 512 at the base 502 for the introduction of the inflation fluid or gas to expand the chamber 514, and also to withdraw the fluid or gas out of the chamber 514. In this manner, the sleeve 500 forms a dilator to be used in dilating a body lumen.

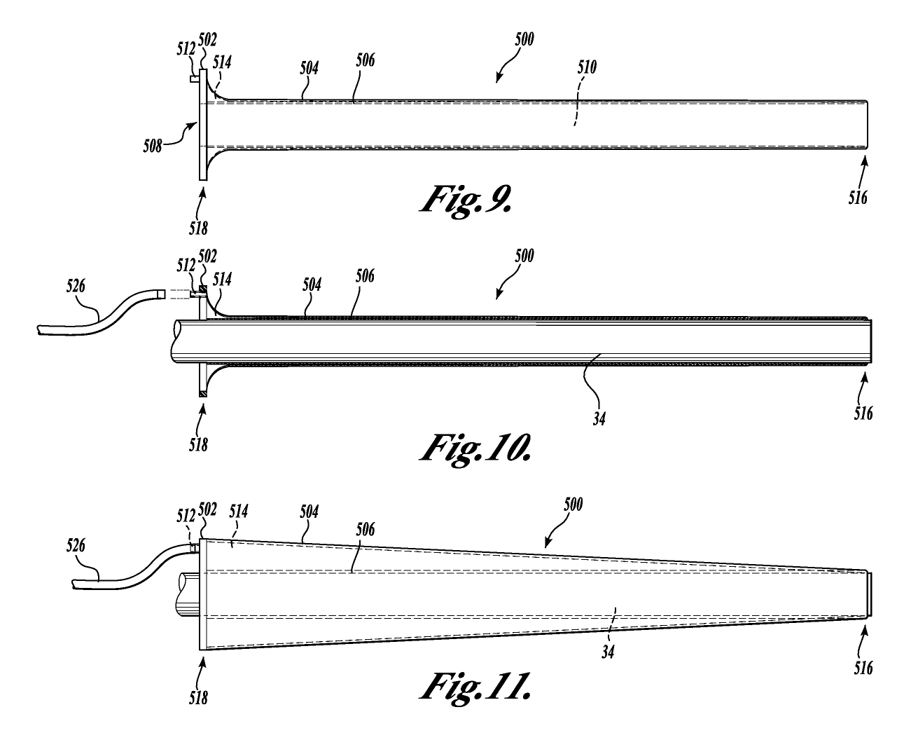

Referring to FIG. 10, the inflatable, but deflated, sleeve 500 is shown attached to the insertion tube 34 of the endoscope 20. The inflatable sleeve 500 is flexible to allow movement of the insertion tube 34. The port 512 at the base 502 of the sleeve 500 may be connected to an inflation/deflation supply hose 526. Hose 526 eventually connects to utilities for inflation and deflation of the sleeve. Alternatively, a source of inflation/deflation may be provided internally through a lumen of the insertion tube 34 that exits the insertion tube 34 and is connected to the supply hose 516 external to the insertion tube 34. Inflation/deflation of sleeve 500 is achieved through operation of the inflation/deflation switches 341, 342 on control handle 24, described above. FIG. 11 illustrates the sleeve 500 when the chamber 514 has been inflated to form a tapered dilator on the insertion tube 34.

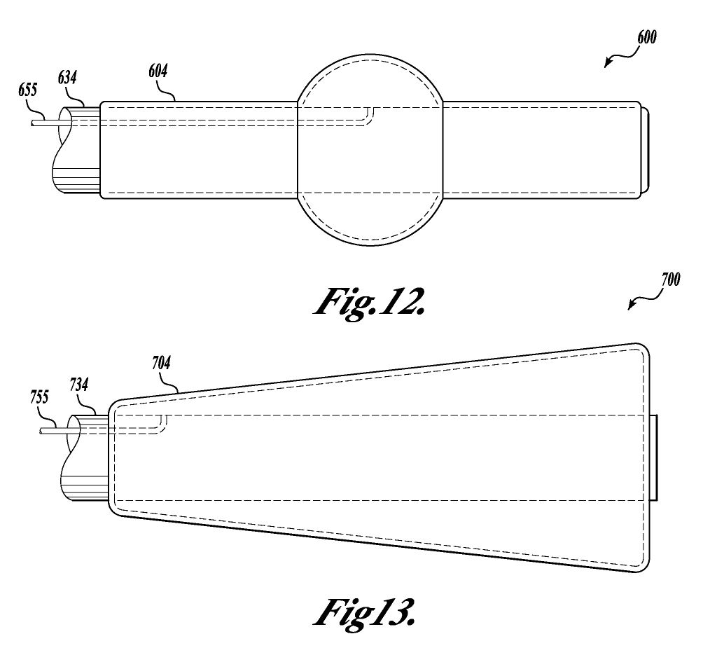

FIG. 12 illustrates another embodiment of a dilator 600 in accordance with an embodiment of the invention. The dilator 600 includes an outer sheath 604 surrounding the end of an insertion tube 634. The outer sheath 604 is expandable. In one embodiment, the outer sheath may be expanded by a fluid, gas, or liquid via the lumen 655 that is delivered to a chamber beneath the outer sheath 604. The outer sheath is formed to expand into a balloon-shaped profile having a rounded configuration.

FIG. 13 is an illustration of another embodiment of a dilator 700 in accordance with one embodiment of the invention. The dilator 700 includes an outer sheath 704 located at the end of the insertion tube 734. The outer sheath 704 defines a chamber therein between the outer sheath 704 and the insertion tube 734. The chamber may be expanded by providing a fluid, either liquid or gas, via lumen 755 to the chamber. The outer sheath 704 is formed to expand into a dilator with a gradually increasing taper upon expansion.

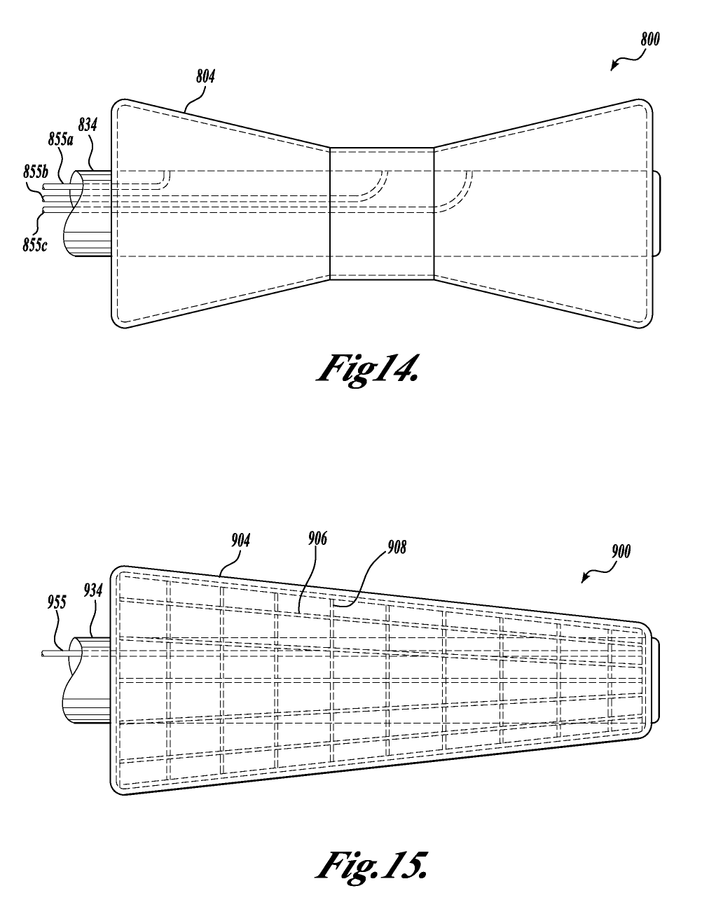

FIG. 14 is an illustration of a dilator 800 in accordance with another embodiment of the invention. The dilator 800 includes multiple sections including a taper from a relatively large diameter to a smaller diameter, a constant diameter section, and a taper from a relatively smaller diameter to a larger diameter. The dilator 800 includes an elastic outer sheath 804 made in a single section or in multiple sections that correspond with the differing profiles of the dilator 800. One way of making the dilator 800 having three separate profiles is by providing three outer sheaths having the desired profile for the particular section. For example, the proximal section can be formed by a sheath that expands into a taper having a gradually decreasing diameter. The proximal section of sheath is circumferentially welded or otherwise attached to the insertion tube 834 in a pressure-tight manner at both ends of the section so as to provide a chamber. A second outer sheath forms the central constant diameter section of the dilator 800. The central section can be circumferentially welded or otherwise attached in a pressure-tight manner to the insertion tube 834 at both ends of the section so as to provide a chamber. A third elastic outer sheath forms the distal section of the dilator 800. The distal section of the dilator 800 tapers from a diameter matching the diameter of the central section to a relatively larger diameter. The distal section can be made from an elastic sheath that is circumferentially welded or otherwise attached in pressure-tight to the insertion tube 834 at both ends to provide a chamber. A first lumen 855a provides a fluid to the chamber of the proximal section of sheath to expand the proximal section of the dilator 800. A second lumen 855b provides fluid to the chamber of the central section of sheath to expand the central section of the dilator 800. A third lumen 855c provides fluid to the chamber of the distal section of sheath to expand the distal section of the dilator 800. Alternatively, the outer sheath 804 can be constructed from a single outer sheath material having a profile wherein the proximal end of the dilator has a relatively larger diameter that tapers to a smaller diameter, a central section that has a constant diameter, and a distal section that tapers from the constant diameter to a relatively larger diameter approximately matching the diameter of the proximal end. The dilator 800 may be used, for example, to dilate the constriction or stricture from both sides. The instrument may be passed to where the constriction is located so that the central portion is at about the location of the constriction and then, expanding the dilator so that the proximal section of the dilator is on one side of the constriction and the distal section of the dilator is on the opposite side of the constriction. In this manner, the constriction may be attempted to be dilated from both sides.

FIG. 15 is an illustration of a dilator 900 in accordance with another embodiment of the invention. The dilator 900 includes an outer expandable sheath 904, which is positioned over the insertion tube 934. A series of radial members 906 and longitudinal members 908 form a “cage” or stent that can be opened to provide a taper from a relatively large diameter to a smaller diameter at the distal end of the insertion tube. The cage or stent may be activated by a control wire 955. This embodiment eliminates the need to have a fluid to cause expansion of the dilator 900. The dilator 900 with mechanical means for expansion may have other shapes as well.

Any one of the dilatable insertion tubes illustrated in FIGS. 5-15 may be used by a physician to perform an endoscopic examination of a body lumen of a patient to examine and to relieve a blockage that may have occurred in the body lumen. To carry out the examination, a patient may be intubated with the insertion tube 34 having a deflated dilator. When the dilator is not in its expanded state, the insertion tube 34 may have a substantially uniform diameter from the proximal end 35 to the distal end 42 to facilitate intubation. If the dilator has a sleeve 500 with a base 502, the base 502 preferably remains exterior to the patient. The physician may next proceed to examine the blockage using the illuminating devices and video imaging capabilities of the endoscope 20.

During or after examination of the blockage, the physician may decide that dilation is needed. Without having to withdraw the insertion tube 34 from the patient, the physician may proceed to dilate the blockage. The physician may perform the dilation procedure without removing the insertion tube 34 from the patient by inflating the dilator or otherwise causing the dilator to expand. According to the operation of the inflation and deflation switches 341 and 342 of a control unit, the physician may create a tapered or nontapered dilator or any other profile. An insertion tube 34 with a tapered dilator may be inflated first, and then, slowly advanced through the blockage. An insertion tube 34 with a nontapered dilator may be positioned in a deflated condition over the blockage, and then, the dilator is slowly inflated to relieve the blockage. Furthermore, at any time, the physician may stop inflation during the dilation, or may even reverse the dilation by deflating the dilator. As fluid or gas is drawn out of the dilator, the outside diameter of the dilator begins to decrease and total deflation may revert the dilator to the condition before inflation. Before, during, and after dilation of the blockage, the physician may view the results without having to withdraw the dilator, as would be the case with a separate instrument dilator.

The use of an endoscope 20 with an insertion tube 34 with a tapered or nontapered dilator and having an expandable outer diameter obviates the need to intubate the patient multiple times, or at least reduces the number of intubations that are necessary with a dilator that is a separate instrument from the endoscope. By providing an endoscope with a dilating capability, the insertion tube may be used for examination of a blockage, as well as for relieving the blockage.

In one embodiment, the endoscope 20, insertion tube 34, control handle 24, and communications conduit 80 may be used only once (a “single-use endoscope”). Thus, with a single-use endoscope, upon completion of a patient examination procedure, the single-use endoscope 20 is disconnected from the control cabinet 28 and discarded. A new single-use endoscope is then connected to the control cabinet 28 for the next examination procedure to be performed on a different patient.

An embodiment of an endoscope includes a control handle having actuatable switches to deliver one or more utilities. The endoscope includes a handle body, wherein at least one switch is disposed on the handle body for delivering at least one utility. The endoscope includes an insertion tube connected to the control handle. The insertion tube has a distal end and a proximal end, wherein the insertion tube is configured to carry one or more utilities to a section of the insertion tube. The endoscope includes an expandable dilator on a section of the insertion tube between the distal end and the proximal end, wherein the dilator is expanded by the delivery of one or more utilities under the control of the control handle. The endoscope may have a dilator that has a tapered outer diameter that extends for a portion of the length of the insertion tube. The endoscope may have a dilator that has a nontapered outer diameter that extends for a portion of the length of the insertion tube. The endoscope may further have an expandable sheath on the insertion tube, wherein the sheath forms the dilator. The endoscope may have a second sheath that covers the insertion tube underneath the expandable sheath. The endoscope may have a sheath defining a chamber that is inflated by a utility. The inflation of the chamber may be controlled by a control unit commanding delivery of a fluid to the chamber. The endoscope may have a dilator that is defined by an inner wall and an outer wall, wherein the inner wall and the outer wall define a pressure-tight chamber that is attached to the insertion tube of the endoscope.

Another embodiment of an endoscope includes a control handle having one or more actuatable switches to deliver one or more utilities. The endoscope includes an insertion tube connected to the control handle. The insertion tube defines a proximal end and a distal end. The insertion tube has a first substantially uniform diameter from the proximal end to the distal end. The endoscope may have an elastic sheath covering the insertion tube, wherein the outer diameter of a section of the insertion tube covered by the sheath may be increased to a relatively larger diameter by a utility controlled by the control unit expanding the elastic sheath. The endoscope may have the diameter of a section of the insertion tube covered by the sheath being tapered from a proximal location to a distal location when the elastic sheath is expanded. The endoscope may have the diameter of a section of the insertion tube covered by the sheath being nontapered from a proximal location to a distal location when the elastic sheath is expanded. The endoscope may have the insertion tube further including a chamber being defined by the elastic sheath and a second sheath located underneath the elastic sheath, wherein the chamber may expand to increase the outer diameter of the insertion tube. The endoscope may have a chamber that can be inflated. The endoscope may have a chamber that includes an inlet for an inflation fluid. The endoscope may have inflation of the chamber being controlled by the control unit.

An embodiment of the invention is a method for dilating a body lumen with an endoscope. The method includes intubating the body lumen with an endoscope having an insertion tube with an expandable outer diameter and dilating the body lumen by increasing the insertion tubers outer diameter. The method may further include examining the effects of dilation with the endoscope without reintubating the endoscope after dilating. The method obviates the need to remove the dilator from the body lumen to enable examining the effects of dilating. The method may further include decreasing the insertion tube's outer diameter after dilating.

Another embodiment is a method for dilating a blockage in a body lumen with an endoscope having an insertion tube. The method includes dilating the blockage with the insertion tube.

An embodiment of an endoscope includes an insertion tube having a proximal end and a distal end and an expandable dilator located on the insertion tube, wherein the dilator may be expanded from a first position to a second position, wherein the dilator is formed from a sheath on the insertion tube. The endoscope may include a dilator that, when expanded, has an outer diameter decreasing towards the distal end of the insertion tube. The endoscope may include a dilator that, when expanded, has an outer diameter increasing towards the distal end of the insertion tube. The endoscope may include a dilator that, when expanded, has a section with an outer diameter decreasing towards the distal end of the insertion tube and a section with an outer diameter increasing towards the distal end of the insertion tube. The endoscope may include a dilator that, when expanded, has a section with an outer diameter decreasing towards the distal end of the insertion tube, a section with an outer diameter increasing towards the distal end of the insertion tube, and a section with a constant diameter. The endoscope may include a dilator that is expanded by a fluid or by a mechanical device, such as a cage or stent.

While the preferred embodiments of the invention have been illustrated and described, it will be appreciated that various changes can be made therein without departing from the scope of the invention. It is therefore intended that the scope of the invention be determined from the following claims and equivalents thereof.

The embodiments of the invention in which an exclusive property or privilege is claimed are defined as follows:

1. An endoscope comprising:
a control handle having controls for delivering at least one utility;
an insertion tube connected to the control handle, wherein the insertion tube has a distal end and a proximal end; and
an expandable dilator on a section of the insertion tube between the distal end and the proximal end.

2. The endoscope of claim 1, wherein the dilator has a tapered outer diameter that extends for a portion of the length of the insertion tube.

3. The endoscope of claim 1, wherein the dilator has a nontapered outer diameter that extends for a portion of the length of the insertion tube.

4. The endoscope of claim 1, wherein the endoscope further comprises an expandable sheath on the insertion tube, wherein the sheath forms the dilator.

5. The endoscope of claim 4, wherein a second sheath covers the insertion tube underneath the expandable sheath.

6. The endoscope of claim 4, wherein the sheath defines a chamber that is inflated by a utility.

7. The endoscope of claim 6, wherein inflation of the chamber is controlled by the control unit commanding delivery of a fluid to the chamber.

8. The endoscope of claim 1, wherein the dilator is defined by an inner wall and an outer wall, wherein the inner wall and the outer wall define a pressure-tight chamber that is attached to the insertion tube of the endoscope.

9. An endoscope comprising:
a control handle having one or more actuatable switches to deliver one or more utilities;
an insertion tube connected to the control handle, wherein the insertion tube defines a proximal end and a distal end; wherein the insertion tube has a first substantially uniform diameter from the proximal end to the distal end; and
a sheath covering the insertion tube, wherein the outer diameter of a section of the insertion tube covered by the sheath may be increased to a relatively larger diameter by a utility controlled by the control unit expanding the elastic sheath.

10. The endoscope of claim 9, wherein the diameter of a section of the insertion tube covered by the sheath is tapered from a proximal location to a distal location when the elastic sheath is expanded.

11. The endoscope of claim 9, wherein the diameter of a section of the insertion tube covered by the sheath is nontapered from a proximal location to a distal location when the elastic sheath is expanded.

12. The endoscope of claim 9, wherein the insertion tube further comprises a chamber defined by the elastic sheath and a second sheath located underneath the elastic sheath, wherein the chamber may expand to increase the outer diameter of the insertion tube.

13. The endoscope of claim 12, wherein the chamber may be inflated.

14. A method for dilating a body lumen with an endoscope, the method comprising:
intubating the body lumen with an endoscope having an insertion tube with an expandable outer diameter and dilating the body lumen by increasing the insertion tube's outer diameter.

15. The method of claim 14, further comprising examining the effects of dilation with the endoscope without reintubating the endoscope after dilating.

16. The method of claim 15, wherein the dilator is not removed from the body lumen to enable examining the effects of dilating.

17. The method of claim 15, further comprising decreasing the insertion tube's outer diameter after dilating.

18. A method for dilating a blockage in a body lumen with an endoscope having an insertion tube, the method comprising dilating the blockage with the insertion tube.

19. An endoscope comprising:
an insertion tube having a proximal end and a distal end; and
an expandable dilator located on the insertion tube, wherein the dilator may be expanded from a first position to a second position.

20. The endoscope of claim 19, wherein when expanded, the dilator has an outer diameter decreasing towards the distal end of the insertion tube.

21. The endoscope of claim 19, wherein when expanded, the dilator has an outer diameter increasing towards the distal end of the insertion tube.

22. The endoscope of claim 19, wherein when expanded, the dilator has a section with an outer diameter decreasing towards the distal end of the insertion tube and a section with an outer diameter increasing towards the distal end of the insertion tube.

23. The endoscope of claim 19, wherein when expanded, the dilator has a section with an outer diameter decreasing towards the distal end of the insertion tube, a section with an outer diameter increasing towards the distal end of the insertion tube, and a section with a constant diameter.

24. The endoscope of claim 19, wherein the dilator is expanded by a fluid.

25. The endoscope of claim 19, wherein the dilator is expanded by a mechanical device.
